(19) United States
(12) Patent Application Publication
(10) Pub. No.: US 2017/0087146 A1
(43) Pub. Date: Mar. 30, 2017

(54) IRINOTECAN HYDROCHLORIDE COMPOSITE PHOSPHOLIPID COMPOSITION, PREPARATION METHOD AND USE THEREOF

(71) Applicants: SHANGHAI INSTITUTE OF MATERIA MEDICA, CHINESE ACADEMY SCIENCES, Pudong Shanghai (CN); SHANGHAI JINGFENG PHARMACEUTICAL CO., LTD., Pudong Shanghai (CN)

(72) Inventors: Yaping LI, Pudong Shanghai (CN); Lingli CHEN, Pudong Shanghai (CN); Zhaolei ZHENG, Pudong Shanghai (CN); Zhiwen ZHANG, Pudong Shanghai (CN); Wangwen GU, Pudong Shanghai (CN)

(21) Appl. No.: 15/124,626

(22) PCT Filed: Mar. 6, 2015
(86) PCT No.: PCT/CN2015/073761

§ 371 Date: Sep. 8, 2016

(30) Foreign Application Priority Data
Mar. 10, 2014 (CN) 201410085842.1

(51) Int. Cl. A61K 31/4745; A61K 9/19; A61K 9/127

(52) U.S. Cl. CPC A61K 31/4745; A61K 9/127; A61K 9/1271; A61K 9/1278; A61K 9/19

(57) ABSTRACT

An irinotecan hydrochloride composite phospholipid composition, preparation method and uses thereof in the preparation of drugs for treating tumors or drug resistant tumors. The composite phospholipid composition comprises irinotecan hydrochloride, composite phospholipid, cholesterol, long-circulating membrane material, surfactant and a buffer medium. The composition improves stability of lipid formulation and the anti-tumor effect of irinotecan hydrochloride, and can overcome multidrug resistance of a tumor.

### TECHNICAL FIELD

The present invention belongs to the field of pharmaceutical preparations, in particular to an irinotecan hydrochloride composite phospholipid composition, method of preparation and the use thereof in the preparation of medicaments for the treatment of tumors or drug-resistant tumors.

### BACKGROUND ART

Irinotecan hydrochloride (Irinotecan, CPT-11) is a semi-synthetic water-soluble camptothecin derivative, and a DNA topoisomerase I (Topo I) inhibitor. Irinotecan and its active metabolite SN-38 cause DNA single-strand breaks by stably binding with DNA-Topo-1 complex, so as to make DNA produce irreversible damage and death. Irinotecan is an effective drug for the treatment of metastatic colorectal cancer, and is still valid for fluorouracil resistant cases, and has a wide anti-tumor spectrum. Phases I and II clinical study results show that the drug has a certain effect on chemotherapy-resistant tumors, such as non-small cell lung cancer, ovarian cancer and cervical cancer. In addition, it has effects on gastric cancer, malignant lymphoma (NHL), breast cancer, small-cell lung cancer, skin cancer and pancreatic cancer.

At present, the products in the domestic market are the injections of irinotecan hydrochloride. Such drug has a strong anticancer activity, but more adverse reactions. The common adverse reactions include anorexia, nausea, vomiting, diarrhea, neutropenia and neutropenia, anemia and thrombocytopenia, alopecia and cholinergic syndrome, and these adverse reactions greatly limits the clinical application of the drug.

Further, the pH-dependent lactone ring in the structure of irinotecan hydrochloride will form an inactive carboxylate form in an alkaline, neutral or weakly acidic (e.g. pH greater than 6.00). Therefore a considerable part of the drug after entering the body will be converted to an inactive carboxylate form, thereby reducing efficacy.

Moreover, multidrug resistance (Multidrug Resistance, MDR) is another major problem of limiting the clinical application of irinotecan hydrochloride. Multidrug resistance refers to that tumor cells produce cross-resistance to other anti-tumor drugs having different structures and mechanisms while being resistant to one anti-tumor drug. Multi-drug resistance is the major cause for the chemotherapy failure of anti-cancer drugs. Multidrug resistance of tumors will greatly reduce the efficacy of irinotecan hydrochloride and other anti-cancer drugs.

Thus, there is a large improvement room for the current dosage forms in the safety, efficacy and overcoming multidrug resistance, which undoubtedly limits its wider application in clinical practice.

In order to enhance the drug targeting, to extend the residence time in vivo, to enhance efficacy and to reduce toxicity, PharmaEngine company developed CPT-11 liposome injection, and currently conducted Phase II clinical trials. The results of Phase I clinical trial conducted in Taiwan that CPT-11 liposome injection has shown good efficacy, tolerability and pharmacokinetic properties when applied to the subjects suffered from advanced refractory tumors.

CN101953792A discloses irinotecan long-circulating nano-liposomes and preparation method thereof. The patent defines that liposome formulation consists of phospholipids, cholesterol, poloxamer 188, polyethylene glycol compounds and irinotecan. The preparation process comprises dissolving phospholipids, cholesterol and poloxamer 188 in ethanol to prepare liposome, dialyzing to remove ammonium sulfate, and then loading the drug. However, there are several problems in this process: (1) poloxamer 188 and polyethylene glycol compounds such as PEG2000 being readily removed while removing ammonium sulfate by dialysis, so as to change the composition ratio; (2) dialysis needs a long time, so that it is difficult to achieve the industrial production; and (3) the external aqueous phase of the liposome is required to be adjusted to 7-7.5, readily rendering worse stability of liposome, inactivation of drug and the like.

CN102485213A and CN103120645 also disclose irinotecan hydrochloride liposomes and preparation methods thereof. However, there are still many problems in the economical efficiency, rationality of the prescription, scalability, easy operation of the process, as well as safety and effectiveness of the drug. None of the above mentions the in vivo or in vitro stability of the liposomal formulations and overcoming the problem of multidrug resistance.

Liposomes as drug carriers not only play a protective effect on the drug, but also improve the targeting of the drug to specific parts of the body, so as to have many superior characteristics in terms of improving efficacy. However, the in vivo and in vitro stability of the liposome limits the clinical application of the liposomes as drug carriers. The instability lies in the following three aspects:
(1) Chemical stability of liposomes: The main component, phospholipid, of liposomes is prone to oxidation and hydrolysis, resulting in decreased fluidity of bilayer membranes, decreased liposomes stability, increasing leakage of the drug;
(2) Physical stability of liposomes: liposomes belong to colloidal dispersion system; phospholipid membrane is a symmetrical bilayer membrane, and there is weak interactions (hydrophobic interactions, van der Waals forces, hydrogen bonding) between the molecules, so that it has thermodynamic instability, mainly as follows: liposome membrane being a dynamic membrane; phospholipid molecules being constantly interchanged; free transmembrane exchange of substances inside and outside the membrane may occur randomly and non-selectively; liposome can spontaneously aggregate and precipitate. In addition, the liposome membrane is generally bilayer gel phase. Phospholipid molecules are tightly arranged; hydrocarbon chain height is ordered; and membrane fluidity is small. Due to changes in temperature, pH and moisture content and other factors, phase change and phase separation thereof often occur. When phase transition of the gel phase→crystal phase (LB→LA) occurs, membrane molecule intervals increase, and fluidity and permeability are notably increased. When phase separation of the gel phase→hexagonal phase (LB→H I or H II) occurs, holes are formed on the membrane, or membrane fusion occurs, so that the drug loaded therein rapidly leaks; and
(3) Biological stability of liposomes: the stability of the liposomes in the blood is the key to play as the drug carrier. There are several disruptive factors: high-density lipoprotein (HDL) is the main component damaging liposomes; apo A-1 protein is easy to fall off from the HDL and combines with liposome phospholipid; HDL and liposomes are prone to the interchange of apo a-1 proteins and phospholipids; liposome membrane forms pores; liposome activates at the same time the complement system in the blood, eventually forming the membrane attack complex (MAC); the liposome membrane appears hydrophilic channel, causing drug leakage and pouring of water and electrolyte, ultimately osmotic cleavage of liposomes; serum albumin and liposome phospholipid are combined to form a complex, so as to reduce the stability thereof; phospholipase in the blood can hydrolyze phospholipids, and the reaction intensity is determined by the phospholipid structure; after liposome enters the body, various conditioning factors, such as antibodies, complements and the like, are combined with lipids, so as to promote the rapid clearance thereof by the reticuloendothelial system.

In addition, the conventional methods for preparation of irinotecan liposomes include the pH gradient method and the ammonium sulfate gradient method, wherein liposomes prepared by the pH gradient method have a poor stability. Usually three dispensing units are designed to improve the formulation stability. However, such repackaged liposome formulations have caused great inconvenience to the production, transportation and clinical use. During the preparation of liposomes by the ammonium sulfate gradient method, ammonium sulfate of the external aqueous phase is removed by dialysis, column chromatography, ultrafiltration or the like. These methods have the problems of a small sample volume, time-consuming, sample dilution, easy to plug the membrane hole and low ultrafiltration efficiency, so as to be suitable for the preparation of a small amount of samples, but not for industrial mass production, thus delaying irinotecan liposome into the clinical process.

### CONTENTS OF THE INVENTION

### Technical Problem

The present inventors have found that, although the disclosed irinotecan liposomes have certain advantages as compared to the commercially available injections, they still have the following defects.
(1) Worse in vitro liposome stability; a specially designed three-bottle or lyophilizing treatment is usually required to resolve the problems of liposome aggregation, easy to leak and the like during the long-term storage.
(2) Worse in vivo liposome stability; after the liposomes enter the body, due to the effect of various factors in the blood, such as albumin, opsonin, antibodies and the like, as well as the lipid phase transition temperature below body temperature, encapsulated drugs leak fast, which greatly reduces the advantages of liposomal formulations, and limits the efficacy thereof
(3) The methods and processes for preparation of the liposomes used therein cannot achieve an industrial production, and the particle size of the resultant liposomes is difficult to control. Moreover, there are the problems of such as heterogeneous distribution, low encapsulation efficiency and poor stability;
(4) The disclosed irinotecan liposomes do not put forward any solutions or coping strategies against multidrug resistance of tumors.

Therefore, with respect to such specific drug-irinotecan hydrochloride, there is a need for looking for a particular formulation and a preparation process thereof aimed at solving the problems of stability, requirements on the industrial production and multidrug resistance of tumors, so as to achieve the object of increasing the formulation stability and efficacy, reducing toxic and side effects and overcoming multidrug resistance of tumors.

### Technical Solution

To solve the above technical problems in the prior art, the present inventors have made extensive and intensive studies, to finally obtain the present invention.

One object of the present invention is to provide a stable clinical irinotecan hydrochloride composite phospholipid composition, which primarily solves the problems of in vivo and in vitro worse stability, being easy to leak and the like, and can greatly improve the stability of liposome formulations and the anti-tumor effect of irinotecan hydrochloride, and overcome multidrug resistance of tumors.

Another object of the present invention is to provide a method for preparing the above irinotecan hydrochloride composite phospholipid composition.

To achieve the above object of the invention, the present invention provides an irinotecan hydrochloride composite phospholipid composition, comprising irinotecan hydrochloride, composite phospholipid, cholesterol, long-circulating membranes, non-ionic surfactant and a buffer medium, wherein said composite phospholipid consists of hydrogenated soybean phospholipids (HSPC), and other lipids.

In the present invention, the irinotecan hydrochloride and the HSPC have a mass ratio of from 1:5 to about 1:50, preferably from 1:5 to about 1:20.

The stability of liposome formulations is directly related to its composition. Lipid formulations formed from different phospholipids have significantly different stabilities, and the lipid formulation formed from a single component phospholipid is extremely unstable. Thus the present invention uses composite phospholipid as the membrane material of the composition. As compared to the liposome formulation composed of a single phospholipid, composite phospholipids can increase the rigidity of the lipid membrane by intermolecular interaction and make the arrangement of phospholipid molecules more closely ordered, which can increase the encapsulation efficiency of the drug, decrease the in vivo and in vitro leakage of the drug, so as to greatly improve the stability thereof.

In the present invention, HSPC and other lipids in the composite phospholipid have a mass ratio 20:1-200:1, preferably 50:1-150:1, more preferably 50:1-100:1.

In the present invention, said other lipids are any pharmaceutically acceptable phospholipids that can be used for preparing liposome formulations, preferably one or more selected from the group consisting of soybean phospholipid (SPC), egg phosphatidylcholine (EPC), hydrogenated egg phosphatidylcholine (HEPC), sphingomyelin (SM), cardiolipin, distearoyl phosphatidylcholine (DSPC), dipalmitoyl phosphatidyl choline (DPPC), dimyristoyl phosphatidylcholine (DMPC), dioleoyl phosphatidyl choline (DOPC), distearoyl phosphatidyl ethanolamine (DSPE), dipalmitoyl phosphatidyl ethanolamine (DPPE), dimyristoyl phosphatidyl ethanolamine (DMPE), dioleoyl phosphatidylethanolamine (DOPE), distearoyl phosphatidyl glycerol (DSPG), dipalmitoyl phosphatidyl glycerol (DPPG), dimyristoyl phosphatidyl glycerol (DMPG) and dioleoyl phosphatidylglycerol (DOPG), more preferably one or more selected from the group consisting of SPC, EPC, HEPC, DSPC and DSP and more preferably DSPC.

In the present invention, the cholesterol as a component of the liposomal formulation acts as a stabilizer, and the amount thereof has a significant influence on the stability and release behavior of the formulation. In the irinotecan hydrochloride composite phospholipid composition of the present invention, HSPC and cholesterol have a mass ratio of about 2:1-about 20:1, preferably from about 2:1-about 10:1, more preferably from about 2:1-about 6:1.

In the present invention, the long-circulating membrane material is used to achieve the long-circulating function of the liposomal formulation, to prolong the circulation time of the drug in the blood, and to increase the drug accumulation at the tumor site, so as to further improve the efficacy and reduce toxicity.

In the irinotecan hydrochloride composite phospholipid composition of the present invention, HSPC and long-circulating membrane material have a mass ratio of about 2:1-about 20:1, preferably about 2:1 to about 10:1.

In the present invention, preferably, the long-circulating membrane material is preferably polyethylene glycol derivatized phospholipid, which is formed by covalently binding polyethylene glycol molecules with reactive groups on phospholipid molecules. Preferably, the polyethylene glycol derivatized phospholipid is one or more selected from the group consisting of polyethylene glycol selected from polyethylene glycol-phosphatidylethanolamine (PEG-PE), polyethylene glycol-dimyristoyl phosphatidyl ethanolamine (PEG-DMPE), polyethylene glycol-dipalmitoyl phosphatidyl ethanolamine (PEG-DPPE), and polyethylene glycol-distearoyl phosphatidyl ethanolamine (PEG-DSPE). The molecular weight of the PEG chain segment in polyethylene glycol derivatized phospholipid is not particularly limited, but preferably has an average molecular weight (number average) of from about 500 to about 5000 Da, more preferably from about 1000 to about 5000 Da, most preferably about 2000 Da. The molecular weight is detected by the gel permeation chromatography (GPC) method.

In the present invention, the non-ionic surfactant results in micelles and emulsions in the lipid suspension. The hydrophobic end thereof is inserted into the twin membrane, and the hydrophilic end makes liposomes highly hydrophilic, so as to prevent the mutual aggregation, fusion and precipitation. It also changes the arrangement and movement of the phospholipid molecules, resulting in an increased longitudinal ordering of the membrane (the close packing of the hydrocarbon chains of phospholipid molecules), a decreased mobility, an increased stability. Moreover, such effect increases with the increase of the concentration thereof. The surfaces of such lipid formulation in the blood are covered by highly hydrophilic albumin to avoid MPS phagocytosis and extend the circulation time of the lipid composition in the blood. Thus the addition of non-ionic surfactant not only can greatly increase the in vivo and in vitro stability of the lipid composition, but also can extend the circulation time of the drug in the body and improve the efficacy. In addition, the non-ionic surfactant, such as Pluronic (Pluronic), natural water-soluble vitamin E (TPGS), 15-hydroxystearate polyethylene glycol (HS15) and the like, can reverse the multidrug resistance of tumors through the following mechanism. Firstly, it can interact with MDR cytomembrane, reduce the microviscosity of the membrane, and suppress Pgp ATPase activity, so as to inhibit the function of the Pgp efflux pump. Secondly, it can inhibit the mitochondrial respiratory chain of MDR cells, reduce the cell membrane potential, induce the release of cytochrome C, increase the reactive oxygen species (ROS) level of cytoplasm, and reduce the ATP content. Thirdly, it can suppress the function of GSH/GST detoxification system. Fourthly, it can increase the pro-apoptotic signals and reduce anti-apoptotic defense of MDR cells. Thus the addition of non-ionic surfactants into the prescription can enhance the sensitivity of the drug-resistant tumors to the drugs, and reverse the multidrug resistance of tumors.

In the irinotecan hydrochloride composite phospholipid composition of the present invention, HSPC and non-ionic surfactant have a mass ratio of about 50:1-about 150:1, preferably about 50:1-about 100:1.

In the present invention, the non-ionic surfactant is one or more selected from the group consisting of Pluronic F68, Pluronic F127, Pluronic P123, Pluronic P85, Pluronic L61, TPGS and HS15.

Studies have shown that hydrolysis of lecithin, saturated soybean phospholipid and phosphatidyl glycerol and the like is affected by pH. The hydrolysates can reduce the pH of lipid suspension, which can promote further hydrolysis of the lipid formulation. Therefore, in the present invention, a buffer medium is added to a suspension of composite phospholipid composition so as to stabilize the pH thereof within the most stable pH range to improve the stability of the composition.

Preferably, the buffer medium in the irinotecan hydrochloride composite phospholipid composition of the present invention can be any pharmaceutically acceptable buffer medium. Preferably, the buffer medium is one or more selected from the group consisting of histidine buffer, glycine buffer solution, phosphate buffer solution and 4-hydroxyethyl piperazine-ethanesulfonic acid (HEPES), and has a concentration range from about 10 to about 50 mM, and a pH of about 5.5 to about 7.5.

The particle size of the composite phospholipid composition will affect its circulation time in the body. Preferably, the irinotecan hydrochloride composite phospholipid composition of the present invention has an average particle size (Z-average particle size) of about 50 to about 200 nm, preferably about 50 to about 120 nm. The particle size was tested by Nano Sizer 90 from Malvern, GB. Preferably, the irinotecan hydrochloride composite phospholipid composition of the present invention may further contain a lyoprotectant. The lyoprotectant was used for lyophilize the resultant composite phospholipid composition to prepare lyophilized powder. In the irinotecan hydrochloride composite phospholipid composition of the present invention, HSPC and lyoprotectant have a mass ratio of about 1:0.1-about 1:5, preferably from about 1:0.5-about 1:4.

Preferably, the lyoprotectant in the present invention is one or more selected from the group consisting of sucrose, lactose, mannitol, trehalose, maltose and the like.

In one preferred embodiment, the irinotecan hydrochloride composite phospholipid composition of the present invention comprises, in parts by weight,

| | |
|---|---|
| HSPC | 100 |
| other phospholipids | 0.5-5, preferably 0.67-2 |
| cholesterol | 5-50, preferably 10-50 |
| long-circulating membrane material | 5-50, preferably 10-50 |
| non-ionic surfactant | 0.67-2, preferably 1-2 |
| irinotecan hydrochloride | 2-20, preferably 5-20 |
| buffer medium | q.s., being stabilized to |
| | have a pH of 5.5-7.5. |

In the aforesaid preferred embodiment, the irinotecan hydrochloride composite phospholipid composition further comprises from about 10 to 500 parts by weight, preferably from about 50 to 400 parts by weight of a lyoprotectant.

The disclosure of the components in the aforesaid preferred embodiment is the same as those above, and will not be repeated herein.

In the irinotecan hydrochloride composite phospholipid composition of the present invention, the pharmaceutical encapsulation efficiency is preferably greater than 80%, so that the lipid formulation can accumulate in the tumor tissues and be less distributed in other normal tissues by the EPR effect, which thereby increases the drug efficacy and reduces the toxicity. In the irinotecan hydrochloride composite phospholipid composition of the present invention, the pharmaceutical encapsulation efficiency is even greater than 85%, more preferably greater than 90%.

The storage stability of a formulation is the key factor affecting the drug efficacy and toxicity. The irinotecan hydrochloride composite phospholipid composition of the present invention is preferably stored at 2-8° C. for stably at least half a year.

In another aspect, the present invention provides a process for preparing the irinotecan hydrochloride composite phospholipid composition by combining the tangential flow ultrafiltration technology with the ammonium sulfate gradient method. Such process can achieve an industrial production, and produce a product having a stable quality in a high efficiency.

The process for preparing the irinotecan hydrochloride composite phospholipid composition comprises the steps of
a. weighing HSPC, other lipids, long-circulating membrane materials and cholesterol in amounts of formula, dissolving them in absolute ethanol to result in an organic phase, pouring the organic phase into an aqueous solution of ammonium sulfate at a concentration of about 100 to about 400 mmol/L, stirring at a high speed (preferably from about 5,000 to about 30,000 rpm), homogenizing, ultrasounding or extruding at a high pressure (preferably from about 10,000 to about 30,000 psi) to form a blank liposome suspension;
alternatively, weighing HSPC, other lipids, cholesterol and long-circulating materials in amounts of formula, dissolving them in tert-butanol, lyophilizing, adding the resultant to an aqueous solution of ammonium sulfate having a concentration of about 100 to about 400 mmol/L to dispense and to form a blank liposome suspension;
b. the external medium of the blank liposome suspension obtained in step a is exchanged about 5 to about 30 times volumes with pure water or aqueous solution of sucrose (having a concentration of 300 mM) through a tangential flow ultrafiltration device (having a membrane molecular weight of 10-100 KDa, a flow rate of 20-400 ml/min, and a pressure of 0-5 bar), to remove ammonium sulfate of the external aqueous phase to establish ammonium sulfate gradient;
c. adding irinotecan hydrochloride into the blank liposome suspension treated by step b, incubating the resultant at a temperature higher than the liposome phase transition temperature (preferably from 37° C. to 70° C.) for 10 min-1 h for drug-loading; and
d. adding a buffer salt and a non-ionic surfactant in a solid form into the drug-loaded liposome suspension, stirring and dissolving, adjusting the pH to 5.5 to 7.5, to obtain an irinotecan hydrochloride composite phospholipid composition;
or replacing the drug-loaded liposome suspension through a tangential flow ultrafiltration device with a pharmaceutically acceptable buffer, then adding a non-ionic surfactant, adjusting the pH to 5.5 to 7.5, to obtain an irinotecan hydrochloride composite phospholipid composition

Further, the preparation process of the present invention may include a step of adding a lyoprotectant after step d.

In the preparation process of the present invention, the microfiltration membrane can be used for sterilizing the resultant by filtration to obtain a sterile preparation after step d or after addition of lyoprotectant.

The ultrasound, high pressure homogenization or extrusion process is to reduce the particle size of the blank liposome suspensions and to control the quality of the product. The tangential flow ultrafiltration process is used to remove ammonium sulfate in the external aqueous phase of the blank liposome suspensions to establish an ionic gradient for drug-loading.

In the above preparation process, the most critical step is to remove ammonium sulfate in the external aqueous phase to produce ammonium sulfate gradient. Currently, the common methods for removing ammonium sulfate in the external aqueous phase include dialysis, column chromatography and ultrafiltration. These three methods have some certain problems, wherein dialysis has a less sample throughput and long duration of dialysis; column chromatography will cause significant dilution of the sample; during the ultrafiltration, membrane pores may be clogged to decrease the efficiency of ultrafiltration, and therefore is suitable only for laboratory processing of a small amount of samples, rather than industrial mass production.

Tangential flow filtration refers to the filtration form in which the fluid flow direction is vertical to the filtration direction. The conventional liquid dead end filtration is mostly microfiltration, comprising the filtration form used for sterilizing, in which the liquid flow direction is consistent with the filtration direction, the thickness of the filter cake layer or gel layer formed on the surface of the filtration membrane is gradually increased, and the flow rate is gradually decreased with the filtration going on. When the filtration medium is the ultrafiltration membrane or microfiltration membrane having fine pore size, and the material liquid has a very high solid content, when performing the dead end filtration, the flow rate will be rapidly decreased. Thus the dead end filtration can be only used for processing a small volume of the material liquid. By using the tangential flow filtration, the liquid flows on the surface of the filtration medium to produce the shear force, to reduce the stack of the filtration cake layer or gel layer, and to ensure the stable filtration rate. Currently it is mainly applied in the cell collection, protein concentration, protein desalting, purification of antibiotics and the like in the pharmaceutical field. The present invention applies it for the removal of ammonium sulfate in the external aqueous phase of liposomes, which involves a significant inventive step.

In the tangential flow filtration device of the present invention, the filtration membrane material is selected from polyether sulfone resin (PES), and triacetate cellulose (CT), and the filtration membrane has a molecular weight of 10-100 KDa, a flow rate of about 20 to about 400 ml/min, and a pressure of 0-5 Bar.

The tangential flow ultrafiltration has the following advantages.

When the tangential flow ultrafiltration is used to replace ammonium sulfate in the external aqueous phase, the sample throughput can reach the industrial scale; moreover, it needs a short period of time, and has a high efficiency and forms a great ionic concentration gradient. The resultant lipid composition has a high encapsulation efficiency which thereby facilitates the industrial production and reduces the production costs. Tangential flow ultrafiltration technology would not alter the properties of the lipid formulation such as the particle size or the distribution thereof, but can make the whole system be in a sealed state; all pipes can be cleaned to prevent the impact of microbes during the operation, so as to provide a guarantee for the sterility of the entire production process, which is critical for the quality control of the injection.

The phase transition temperature refers to the temperature at which a lipid interconverts between the gel state and crystal state. When incubating at a temperature higher than the lipid phase transition temperature, the lipid membrane will have an enhanced permeability, and irinotecan driven by the ion gradient is more membrane-permeable and aggregates in the internal aqueous phase of liposomes.

The present invention further provides use of said irinotecan hydrochloride composite phospholipid composition in the preparation of the drugs for the treatment of tumors, particularly drug-resistant tumors, wherein the tumor is colorectal cancer, non-small cell lung cancer, ovarian cancer, cervical cancer, stomach cancer, malignant lymphoma, breast cancer, skin cancer or pancreatic cancer.

The present invention has the following advantages over the prior art.

The present invention uses composite phospholipid as one component of the lipid composition, which can change the structure of the lipid membrane and enhance the rigidity of the membrane by the interaction between two phospholipids as compared to a single phospholipid, so as to make the arrangement of phospholipid molecules more closely ordered, reduce the permeability of the lipid membrane and greatly decrease the pharmaceutical leakage during storage or in vivo circulation, thus helping to improve the efficacy. Such technological advantage is not reported in other related patent documents.

The addition of non-ionic surfactant in the formulation of the present invention makes the presence of micelles in the lipid suspension. The hydrophobic end is inserted into the bimolecular membrane, and the hydrophilic end makes the lipid formulation highly hydrophilic, so as to prevent the mutual aggregation, integration and precipitation between lipid formulations. It also changes the arrangement and movement of the phospholipid molecules, resulting in an increased longitudinal ordering of the membrane (the close packing of the hydrocarbon chain of phospholipid molecules), a decreased mobility, and an increased stability. The surfaces of such lipid formulation in the blood are covered by highly hydrophilic albumin to avoid MPS phagocytosis and extend the circulation time of the composite phospholipid composition in the blood, which can greatly increase the physical stability and biological stability of composite phospholipid compositions. Moreover, the surfactants, such as Pluronic, HS15, TPGS, may also enhance the sensitivity of drug-resistant tumors to the drugs, and reverse the multidrug resistance of tumors.

The buffer medium can maintain the pH of the composite phospholipid composition in a certain range, reduce oxidative hydrolysis of the composite phospholipid materials and improve the chemical stability of the composite phospholipid composition.

The composite phospholipid composition is used as the carrier of irinotecan hydrochloride, and the drug is encapsulated in the composite phospholipid composition, which can significantly improve the stability of the drug in the body, maintain its active lactone ring structural form, and better play the anti-cancer role. The irinotecan hydrochloride composite phospholipid composition belongs to the nano-preparation category, and can significantly prolong the circulation time of the drug in the blood, improve its biodistribution, increase the drug accumulation at the tumor site, improve the efficacy, reduce the toxic and side effect, so as to improve the therapeutic index.

The irinotecan hydrochloride composite phospholipid composition of the present invention has an average particle size of from about 50 to about 200 nm, and can effectively penetrate the tumor blood vessels, aggregate at the tumor site by enhanced permeation and retention effect (EPR effect) to achieve passive targeting.

The irinotecan hydrochloride composite phospholipid composition of the present invention is prepared by using the novel tangential flow ultrafiltration technology in combination with the ammonium sulfate gradient method, which is easier to achieve the industrial production than the current preparation method, and can solve the problems in the prior art of large particle size and inhomogeneity, and better control the product quality. One could prepare the irinotecan hydrochloride composite phospholipid composition having an encapsulation rate greater than 80% through mixing a blank liposome with irinotecan hydrochloride for incubation by using the ammonium sulfate gradient. The method is easy to operate and provides a convenient quick and easy way for the clinical application of the formulation.

### DESCRIPTION OF THE DRAWINGS

FIG. 1 shows the particle size distribution diagram of the irinotecan hydrochloride composite phospholipid composition prepared according to Example 1 of the present invention.

FIG. 2 shows the zeta potential diagram of the irinotecan hydrochloride composite phospholipid composition prepared according to Example 2 of the present invention.

FIG. 3 shows the in vitro release test results of the irinotecan hydrochloride composite phospholipid composition prepared according to Example 1 of the present invention and the liposome prepared according to Comparison Example 1 of the present invention.

FIG. 4 shows the efficacy test results of the irinotecan hydrochloride composite phospholipid composition prepared according to Example 1 of the present invention and the liposome prepared according to Example 2 of the present invention, by using saline as a control.

### EMBODIMENTS

The present invention is further illustrated by combining with the following examples, and the following embodiments only describe the present invention by way of examples. However, these examples are not meant to be any limitation to the present invention. Obviously, those ordinary skilled in the art may make various changes and modifications within the scope and spirit of the present invention. It should be understood that the present invention is intended to cover the modifications and changes in the claims.

Reagents and Drugs

Soybean lecithin (Shanghai Taiwei Pharmaceutical Co., Ltd.); HSPC (Shanghai Advanced Vehicle Technology Co., Ltd.); PEG-DSPE (Shanghai Advanced Vehicle Technology Co., Ltd.); PEG-PE (Shanghai Advanced Vehicle Technology Co., Ltd.); PEG-DPPE (Shanghai Advanced Vehicle Technology Co., Ltd.); sphingomyelin (Shanghai Ziyi Reagent Factory); egg lecithin (Shanghai Advanced Vehicle Technology Co., Ltd.); cholesterol (Nanjing Xinbai Pharmaceutical Co., Ltd.); sephadex G-50 (GE, USA); irinotecan hydrochloride (Shanghai Acebright Pharmaceuticals Group Co., Ltd.).

### Example 1 Preparation of Irinotecan Hydrochloride Composite Phospholipid Composition

1.2 g of HSPC, 0.012 g of DSPC, 0.3 g of cholesterol and 0.4 g of PEG2000-DSPE were dissolved by ultrasounding in 1.5 ml of absolute ethanol, and then poured into 30 ml of 250 mM aqueous solution of ammonium sulfate preheated to 65° C., stirred at high speed (at a rotary rate of 20,000 rpm) to obtain the primary product; the primary product was then homogenized four times under a high pressure of 20,000 psi. Subsequently, the external medium was exchanged 10 volumes with ultrapure water using tangential flow ultrafiltration system (having a molecular weight of 30 kDa membrane, a flow rate of 200 ml/min, and a pressure of 1 bar) to remove ammonium sulfate of the external aqueous phase, to obtain a blank liposome suspension. The resultant blank liposome suspension and the aqueous solution of irinotecan hydrochloride (having a concentration of 10 mg/ml) were mixed at a drug/HSPC ratio of 1:10 by weight, incubated at 65° C. for 30 min. Then 0.012 g of F68, 4.3 g of sucrose and 0.065 g of histidine were added, and the pH was adjusted to 6.0, so as to obtain the irinotecan hydrochloride composite phospholipid composition.

### Example 2 Preparation of Irinotecan Hydrochloride Composite Phospholipid Composition

1.5 g of HSPC, 0.02 g of soya lecithin, 0.15 g of cholesterol, and 0.15 g of PEG2000-DSPE were dissolved in 1.5 ml of absolute ethanol, then poured into 30 ml of 200 mM aqueous solution of ammonium sulfate preheated to 65° C., stirred at high speed (at a rotary rate of 25,000 rpm) to obtain the primary product; the primary product was then homogenized four times at a high pressure of 15,000 psi-. Subsequently, the external medium was exchanged 20 volumes with 300 mM sucrose solution using tangential flow ultrafiltration system (having a molecular weight of 30 kDa membrane, a flow rate of 300 ml/min, and a pressure of 1.5 bar) to remove ammonium sulfate of the external aqueous phase, to obtain a blank liposome suspension. The resultant blank liposome suspension and irinotecan hydrochloride solution (having a concentration of 10 mg/ml) were mixed in a drug/HSPC weight ratio of 1:20 by weight, incubated at 55° C. for 1 h; then the unencapsulated drug was removed while the external medium was exchanged with sucrose/histidine buffer pH6.5(300 mM sucrose,10 mM histidine) using tangential flow ultrafiltration system. Then 0.02 g of HS15 was added, stirred and dissolved, aseptically filtrated and subpacked and stored at 4° C. for later use.

### Example 3 Preparation of Irinotecan Hydrochloride Composite Phospholipid Composition

1.2 g of HSPC, 0.024 g of egg yolk lecithin, 0.12 g of cholesterol, and 0.4 g of PEG2000-DPPE were dissolved in 1.5 ml of absolute ethanol, then poured into 30 ml of 250 mM aqueous solution of ammonium sulfate preheated to 65° C., stirred at a high speed (at a rotational speed of 20,000 rpm), to obtain the primary product. The resultant primary product was then extruded with Polycarbonate film having a pore size of 100 nm four times. The external medium was exchanged 5 volumes with 300 mM sucrose solution using the tangential flow ultrafiltration system (having a membrane molecular weight of 30 kDa, a flow rate of 100 ml/min, and a pressure of 1.5 bar) to remove ammonium sulfate of the external aqueous phase, so as to obtain a blank liposome suspension. The resultant blank liposome suspension and the aqueous solution of irinotecan hydrochloride (having a concentration of 5 mg/ml) were mixed in a drug/HSPC ratio of 1:5 by weight, incubated at 60° C. for 10 min; and then using tangential flow ultrafiltration system, the unencapsulated drug was removed while the external aqueous phase was exchanged with 300 mM sucrose, 20 mM phosphate buffer solution (pH=7.4). Then 0.02 g of TPGS was added and dissolved to obtain the product.

### Example 4 Preparation of Irinotecan Hydrochloride Composite Phospholipid Composition

1.5 g of HSPC, 0.015 g of DSPG, 0.4 g of cholesterol, and 0.4 g PEG2000-DSPE were dissolved in 1.5 ml of absolute ethanol, then poured into 30 ml of 250 mM aqueous solution of ammonium sulfate preheated to 65° C., stirred at a high speed (at a rotary rate of 20,000 rpm) to obtain the primary product. The resultant primary product was then extruded with Polycarbonate film having a pore size of 100 nm four times. The external medium was exchanged 5 volumes with 300 mM sucrose solution using the tangential flow ultrafiltration system (having a membrane molecular weight of 30 kDa, a flow rate of 100 ml/min, and a pressure of 1.5 bar) to remove ammonium sulfate of the external aqueous phase, so as to obtain a blank liposome suspension. The resultant liposome suspension and the aqueous solution of irinotecan hydrochloride (having a concentration of 5 mg/ml) were mixed in a drug/HSPC ratio of 1:10 by weight, incubated at 60° C. for 10 min; and then using tangential flow ultrafiltration system, the unencapsulated drug was removed while the external aqueous phase was exchanged with 300 mM sucrose, 20 mM phosphate buffer solution (pH=7.4). Then 0.015 g of Poloxamer F68 was added and dissolved to obtain the product.

### Example 5 Preparation of Irinotecan Hydrochloride Composite Phospholipid Composition Lyophilized Powder

1.6 g of HSPC, 0.032 g of HEPC, 0.16 g of cholesterol, and 0.5 g of PEG2000-DMPE were dissolved in 5 ml of tertiary butanol, and then lyophilized in a lyophilizer. Then 30 ml of 200 mM ammonium sulfate solution was added, hydrated and ultrasounded until the mixture is semitransparent. The external medium was exchanged 15 volumes with ultrapure water using the tangential flow ultrafiltration system (having a membrane molecular weight of 10 kDa, a flow rate of 200 ml/min, and a pressure of 1.5 bar) to remove ammonium sulfate of the external aqueous phase to obtain a blank liposome suspension. 30 ml of the resultant blank liposome suspension and 10 ml of the irinotecan hydrochloride solution (containing 80 mg of irinotecan hydrochloride) were mixed and incubated at 60° C. for 1 h. Then 0.008 g of Pluronic F127, 0.03 g of glycine, 0.2 g of sucrose, 0.5 g of mannitol and 1 g of lactose were added and dissolved by stirring, and after adjusting the pH to 7.4, the mixture was lyophilized in a lyophilizer to obtain the irinotecan hydrochloride composite phospholipid composition lyophilized powder.

### Comparison Example 1

### Preparation of the Current Liposomes of Irinotecan Hydrochloride

According to the formulations and method disclosed in CN101953792A, the preparation is disclosed as follows.

3 g of soy lecithin, 1 g of cholesterol, 0.6 g of Poloxamer 188, and 0.1 g of Vitamin E were weighed and dissolved in 1.5 ml of absolute ethanol, then injected under the condition of a water bath at 55° C. into 30 ml of ammonium sulfate solution (200 mM) in which 0.3 g of PEG-DSPE was dissolved, stirred isothermally for 1 h under nitrogen conditions. The resultant long-circulating blank liposome was dialyzed in saline overnight. Sodium hydroxide was used to adjust the pH of external phase to 7.4, and then 30 ml of irinotecan hydrochloride solution (10 mg/ml) was added, incubated at 55° C. for 10 min, sterilized by filtering, and subpacked in a vial by 4 ml/bottle.

### Comparison Example 2

### Preparation of the Current Liposomes of Irinotecan Hydrochloride

According to the formulations and method disclosed in CN103120645A, the preparation is disclosed as follows.

1 g of HSPC and 0.25 g of cholesterol were dissolved in a suitable amount of absolute ethanol to obtain a lipid solution, and then mixed with 100 ml of 250 mM ammonium sulfate solution. Ethanol was removed under a reduced pressure to obtain the crude blank liposome products. Then a high-pressure homogenizer was used to homogenize at 1000 bar for 5 cycles, and an extrusion equipment was used to extrude the liposome to control the particle size (2 sheets of 0.1 μm extrusion membrane were laid out on the extruder, and extrusion was carried out 5 times). 0.1 g of DSPE-PEG2000 aqueous solution was added, stirred and incubated for 20 min. Ultrafiltration device was used to dialyze the liposome, in which water for injection was uninterruptedly replenished to obtain the liposome.

An aqueous solution of irinotecan hydrochloride was formulated with water for injection, and added to the above blank liposome dispersion having an ionic gradient according to the weight ratio 1:3.5 of irinotecan hydrochloride to HSPC, heated and stirred at 60° C., incubated for 20 minutes to obtain the drug-loaded liposomes. Tangential flow ultrafiltration apparatus was used to remove the non-encapsulated drug, and concentrate the sample to about 50 ml. 0.45 g of sodium chloride was added to regulate the osmotic pressure, followed by the steps of adjusting the drug concentration, setting the volume precisely, filtering and sterilizing with 0.22 μm filter membrane, filling nitrogen and encapsulating the product in a vial to obtain the irinotecan hydrochloride liposome injection.

Performance Testing

Particle Size and Distribution Test

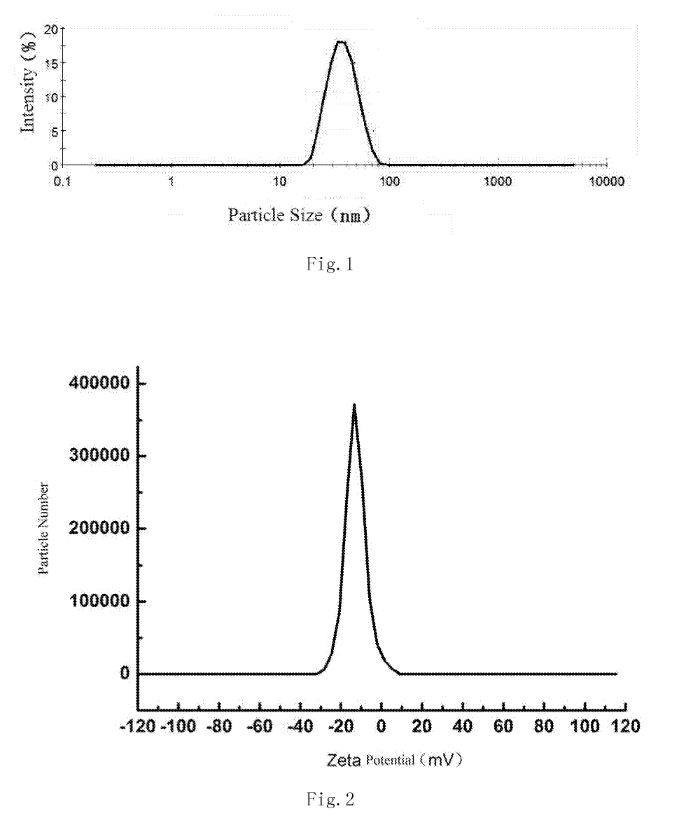

The irinotecan hydrochloride composite phospholipid composition obtained in Example 1 was diluted with water, and the particle size and distribution were tested by the particle size analyzer (Model: Nano Sizer 90, from Malvern). The result was shown in FIG. 1, wherein Z-average particle size was 71.53 nm; the polydispersity index was 0.107, indicating that the particles were evenly distributed.

The particle size and distribution results of the composite phospholipid compositions in other examples are shown in Table 1.

TABLE 1

| | Example 2 | Example 3 | Example 4 | Example 5 |
|---|---|---|---|---|
| Particle size(nm) | 79 | 78.43 | 92.48 | 84.98 |
| Polydispersity index | 0.192 | 0.225 | 0.244 | 0.238 |

Determination of Zeta Potential

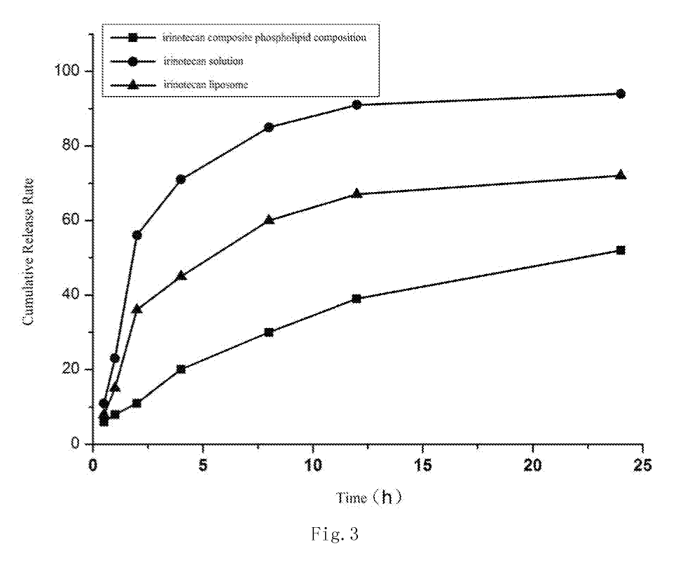

Vinorelbine tartrate long-circulating liposome prepared in Example 2 was taken, and its zeta potential was determined with nanosizer 90. The result was shown in FIG. 2, and the zeta potential was −12.4 mv.

Determination of Encapsulation Efficiency

The irinotecan hydrochloride composite phospholipid compositions prepared in Examples 1-5 were taken for the determination of encapsulation efficiency.

Chromatographic conditions: chromatographic column Waters SunFire® C18 column (4.6 mm×250 mm, 5 μm); mobile phase was acetonitrile −26 mmol/L sodium dihydrogen phosphate solution (containing 8 mmol/L octyl sodium sulfonate) (32:68); the flow rate was 1 ml/min; the column temperature was 40° C.; the wavelength was detected to be 254 nm; the injection volume was 20 μl.

A suitable amount of fully swollen Sephadex G-50 sephadex was used to prepare a gel column (30 cm×1 cm), 0.5 ml of the precise amount of the irinotecan hydrochloride composite phospholipid composition was weighed, fed to the column and eluted with PBS (PH=7.4). 14 ml of the fractions containing the composition was collected, placed in a measuring flask of 50 ml, volumed with methanol and homogeneously shaken. 0.5 ml precisely weighed was placed in a flask of 10 ml, and volumed with acidic methanol. The dose W encapsulated in the composite phospholipid composition was determined by HPLC. 0.5 ml of irinotecan hydrochloride composite phospholipid was placed in a flask of 50 ml, operated by the same process to determine the total dose W0 in the clinical drug-loaded nano-formulation. By calculating, the clinical drug-loaded nano-formulation of irinotecan hydrochloride had an average encapsulation efficiency of 91.27%.

TABLE 2

| | Example 1 | Example 2 | Example 3 | Example 4 | Example 5 |
|---|---|---|---|---|---|
| Encap- | 95.6 | 97.6 | 93.1 | 95.9 | 96.3 |
| sulation | | | | | |
| efficiency | | | | | |

In Vitro Release Assay

The irinotecan hydrochloride composite phospholipid composition in Example 1, irinotecan liposome and irinotecan hydrochloride solution in Comparison Example 1 were taken for the in vitro release assay. The irinotecan hydrochloride solution was formulated by weighing a certain amount of irinotecan hydrochloride and formulating with ultrapure water to a solution having a concentration of 2 mg/ml.

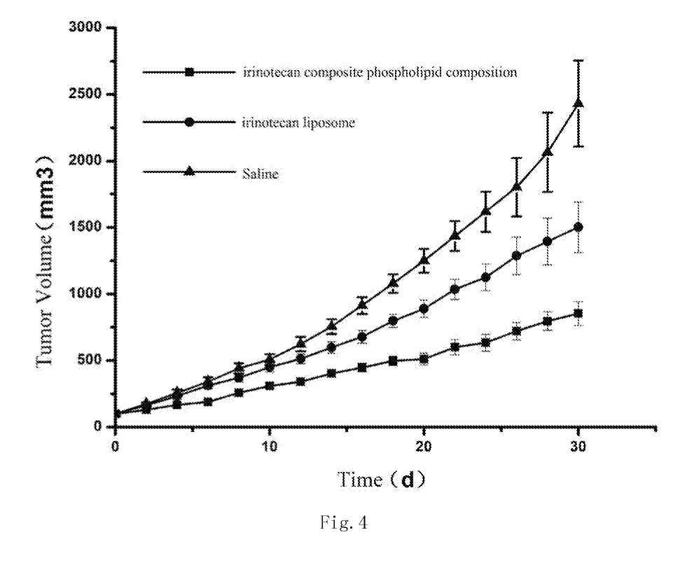

1 ml of the irinotecan composite phospholipid composition or irinotecan liposome was precisely weighed and added to a dialysis bag (having a molecular weight of 8000-14,000 Da), which were tightened at both ends, then placed in a conical flask containing 20 ml of release medium (phosphate buffer having a pH of 7.4), oscillated at constant rate (100 r/min) at (37.0±0.5°) C. Samples were taken at 0.5, 1, 2, 4, 8, 12 and 24 h, and determined to calculate the cumulative release rate (%). Meanwhile, the release of the irinotecan solution was examined. Release curve was obtained by plotting the cumulative release rate (Q) vs. time (t), and was shown in FIG. 3.

FIG. 3 shows that, as compared to the irinotecan liposome, the irinotecan composite phospholipid composition was released more slowly, and the cumulative release rate at 24 h was 52%, indicating that the composite phospholipid composition of the present invention had a better stability than the liposome in Comparison Example 1.

Stability Test

The irinotecan hydrochloride composite phospholipid composition in Example 1 and irinotecan hydrochloride liposome in Comparison Example 2 were taken for the stability test.

The irinotecan hydrochloride composite phospholipid composition in Example 1 was placed at 2-8° C.; samples were taken at 0, 1, 2, 3 and 6 months to determine the quality indexes, such as total content of irinotecan hydrochloride, related substances (residual synthetic material, intermediate, side product, possible degradation products and the like were collectively called as related substances), encapsulation efficiency, particle size and the like. The irinotecan hydrochloride liposome in Comparison Example 2 was placed under the same conditions, and then directly sampled to determine the aforesaid quality indexes.

As can be seen from Table 3, the total content of irinotecan hydrochloride decreased by 5.6%, and the related substances increased by 3.53% after the irinotecan liposome in Comparison Example 2 was placed at 2-8° C. for 6 months. After the irinotecan hydrochloride composite phospholipid composition of the present invention was placed for 6 months, each quality index had no obvious change as compared to that at 0 month, indicating that the irinotecan hydrochloride composite phospholipid composition of the present invention has a greatly increased stability over the current formulations and has a better clinical application value.

TABLE 3
Stability of the current liposome and the irinotecan hydrochloride
phospholipid composition of the present invention

| | | | | | | irinotecan hydrochloride | | | | |
|---|---|---|---|---|---|---|---|---|---|---|
| | | | | | | phospholipid composition of the present | | | | |
| | Current liposome | | | | | invention | | | | |
| Quality | 0 | 1 | 2 | 3 | 6 | 0 | 1 | 2 | 3 | 6 |
| indexes | month | month | months | months | months | month | months | months | months | months |
| Total content | 4.98 | 4.90 | 4.84 | 4.76 | 4.70 | 1.66 | 1.65 | 1.64 | 1.65 | 1.63 |
| (mg/ml) | | | | | | | | | | |
| Related | 0.26 | 0.92 | 1.19 | 1.82 | 3.79 | 0.24 | 0.28 | 0.29 | 0.33 | 0.41 |
| substances (%) | | | | | | | | | | |
| Encapsulation | 95.6 | 96.3 | 95.2 | 95.7 | 95.8 | 98.1 | 98.5 | 98.3 | 98.4 | 98.6 |
| efficiency (%) | | | | | | | | | | |
| Particle size | 93.73 | 93.87 | 94.71 | 96.88 | 99.80 | 75.53 | 77.81 | 75.77 | 78.37 | 77.58 |
| (nm) | | | | | | | | | | |

Pharmacokinetics Test

8 healthy male SD rats (Source: Shanghai Experimental Animal Center) (weighing 200-220 g) were randomly divided into 2 groups. Caudal vein was injected respectively with the irinotecan hydrochloride phospholipid composition, irinotecan liposome and irinotecan injection liquid in Example 1 and Comparison Example 2 at a dose of 10 mg/kg by caudal vein administration. 0.3 ml of blood was taken from orbital venous plexus in rats at 0.083 h, 0.25 h, 0.5 h, 1 h, 2 h, 4 h, 6 h, 8 h and 24 h, placed in a heparinized tube, centrifuged at 10,000 rpm for 10 min. 100 μl of plasma was taken, and then 10 μl of 10% formic acid and 500 μl of methanol were added thereto and vortexed for 1 min, placed at −20° C. for 1 h to precipitate proteins, and then centrifuged at 20,000 g for 10 min. Supernatant was taken and used to measure the irinotecan concentration in the blood. Pharmacokinetic parameters were analyzed and processed by WinNonlin Professional v6.3 (Pdayarsight, USA) software using non-compartmental models.

TABLE 4

| | | Composite phospholipid | | |
|---|---|---|---|---|
| Parameter | Unit | composition | Liposome | Injection liquid |
| Kₑₗ | L/kg | 0.193 ± 0.0183* | 0.501 ± 0.0647 | 0.496 ± 0.0546 |
| t_{1/2} (h) | hr | 12.61 ± 0.358* | 1.39 ± 0.176 | 1.41 ± 0.174 |
| AUC₀₋₂₄ | hr * ng/mL | 823721 ± 33816* | 19885 ± 3569 | 3478 ± 273 |
| AUC_{0-∞} | hr * ng/mL | 823732 ± 33823* | 19897 ± 3580 | 3491 ± 281 |

Note:
Kₑ₁ is the elimination rate constant;
t_{1/2} is the half-life;
AUC is the area under the curve of plasma concentration vs. time.

As can be seen from Table 4, the half life and AUC of the irinotecan hydrochloride composite phospholipid composition of the present invention are 9.06-fold and 41-fold of the irinotecan liposome in Comparison Example 2, indicating that the irinotecan hydrochloride composite phospholipid composition of the present invention has a better in vivo stability over the liposome, greatly extends the biological half-life of irinotecan hydrochloride and increases the bioavailability of the drug.

Antitumor Activity Test

Balb/c nude mice (purchased from Shanghai Experimental Animal Center) adapted to the environment 5d. MCF-7/ADR cells at the logarithmic growth phase were digested to obtain 1×10⁸/ml cell suspension. 0.1 ml of cell suspension was subcutaneously injected to right forelimb Balb/c nude mice to establish the tumor-bearing models. Until the average mouse tumor volume grew to 50-100 mm³, the mice were randomly divided into three groups each of which included 10 mice. Each group was injected by caudal vein at the first, fourth and seventh days, and the administration dose was 20 mg/kg of CPT-11 composite phospholipid composition in Example 1, 20 mg/kg of CPT-11 in Comparison Example 2 and saline (control group). The long diameter (a) and minor diameter (b) of each mouse were measured with a caliper, and the tumor volume was calculated by the formula (a×b²)/2.

As can be seen from FIG. 4, the irinotecan hydrochloride composite phospholipid composition of the present invention and the irinotecan hydrochloride liposome both have a better inhibitory effect on drug-resistant breast cancer of nude mouse. The tumor volume of each dose group is significantly decreased (P<0.05, 0.01) over the control group (i.e. physiological saline group). Moreover, the composite phospholipid in the same dosage has a better anti-tumor effect (P<0.05) over the irinotecan hydrochloride phospholipid group in Comparison Example 2, indicating that the irinotecan hydrochloride composite phospholipid composition of the present invention can reverse the tumor resistance to a certain extent.

1-16. (canceled)

17. An irinotecan hydrochloride composite phospholipid composition, comprising:
irinotecan hydrochloride;
composite phospholipid;
cholesterol;
long-circulating membrane material;
nonionic surfactant; and
a buffer medium;
wherein the composite phospholipid consists of hydrogenated soybean phospholipids (HSPC) and other lipids.

18. The irinotecan hydrochloride composite phospholipid composition according to claim 17,
wherein the irinotecan hydrochloride and the HSPC have a mass ratio of 1:5-1:50.

19. The irinotecan hydrochloride composite phospholipid composition according to claim 17,
wherein the HSPC and the other lipids in the composite phospholipid have a mass ratio of 20:1-200:1.

20. The irinotecan hydrochloride composite phospholipid composition according to claim 17,
wherein said other lipids are one or more selected from the group consisting of soybean phospholipid (SPC), egg phosphatidylcholine (EPC), hydrogenated egg phosphatidylcholine (HEPC), sphingomyelin (SM), cardiolipin, distearoyl phosphatidylcholine (DSPC), dipalmitoyl phosphatidyl choline (DPPC), dimyristoyl phosphatidylcholine (DMPC), dioleoyl phosphatidyl choline (DOPC), distearoyl phosphatidyl ethanolamine (DSPE), dipalmitoyl phosphatidyl ethanolamine (DPPE), dimyristoyl phosphatidyl ethanolamine (DMPE), dioleoyl phosphatidylethanolamine (DOPE), distearoyl phosphatidyl glycerol (DSPG), dipalmitoyl phosphatidyl glycerol (DPPG), dimyristoyl phosphatidyl glycerol (DMPG) and dioleoyl phosphatidylglycerol (DOPG).

21. The irinotecan hydrochloride composite phospholipid composition according to claim 17,
wherein the HSPC and the cholesterol have a mass ratio of 2:1-20:1.

22. The irinotecan hydrochloride composite phospholipid composition according to claim 17,
wherein the HSPC and the long-circulating membrane material have a mass ratio of 2:1-20:1.

23. The irinotecan hydrochloride composite phospholipid composition according to claim 17,
wherein the long-circulating membrane material is polyethylene glycol derivatized phospholipids formed by covalently binding polyethylene glycol molecules with reactive groups on phospholipid molecules.

24. The irinotecan hydrochloride composite phospholipid composition according to claim 23,
wherein the polyethylene glycol derivatized phospholipid is one or more selected from the group consisting of polyethylene glycol selected from polyethylene glycol-phosphatidylethanolamine (PEG-PE), polyethylene glycol-dimyristoyl phosphatidyl ethanolamine (PEG-DMPE), polyethylene alcohol-dipalmitoyl phosphatidyl ethanolamine (PEG-DPPE), polyethylene glycol-distearoyl phosphatidyl ethanolamine (PEG-DSPE).

25. The irinotecan hydrochloride composite phospholipid composition according to claim 17,
wherein the HSPC and the nonionic surfactant have a mass ratio of 50:1-150:1.

26. The irinotecan hydrochloride composite phospholipid composition according to claim 17,
wherein the non-ionic surfactant is one or more selected from the group consisting of Pluronic F68, Pluronic F127, Pluronic P123, Pluronic P85, Pluronic L61, TPGS and HS15.

27. The irinotecan hydrochloride composite phospholipid composition according to claim 17,
wherein said buffer medium is one or more selected from the group consisting of histidine buffer, glycine buffer, phosphate buffer and 4-hydroxyethyl piperazine-ethanesulfonic acid (HEPES) buffer, and the concentration thereof ranges from about 10 to about 50 mM, and the pH thereof is 5.5-7.5.

28. The irinotecan hydrochloride composite phospholipid composition according to claim 17,
wherein the irinotecan hydrochloride composite phospholipid composition has a Z-average particle size of 50-200 nm

29. The irinotecan hydrochloride composite phospholipid composition according to claim 17,
wherein the HSPC and lyoprotectant have a mass ratio of 1:0.1-1:5; and
wherein the lyoprotectant is one or more selected from the group consisting of sucrose, lactose, mannitol, trehalose, maltose and the like.

30. The irinotecan hydrochloride composite phospholipid composition according to claim 17, wherein: in parts by weight,
HSPC is present at 100 parts by weight;
other phospholipids are present at 0.5-5 parts by weight;
cholesterol is present at 5-50 parts by weight;
long-circulating membrane material is present at 5-50 parts by weight
non-ionic surfactant is present at 0.67-2 parts by weight;
irinotecan hydrochloride is present at 2-20 by weight; and
buffer medium is present at q.s., being stabilizing to have a pH of 5.5-7.5.

31. The irinotecan hydrochloride composite phospholipid composition according to claim 30,
wherein the lyoprotectant is present at about 10 to 500 parts by weight.

32. The irinotecan hydrochloride composite phospholipid composition according to claim 17,
wherein the pharmaceutical encapsulation efficiency of the composition is greater than 80%.

33. A process for preparing the irinotecan hydrochloride composite phospholipid composition according to claim 17, comprising the steps of:
a. weighing HSPC, other lipids, long-circulating membrane materials and cholesterol in amounts of formula, dissolving them in absolute ethanol to result in an organic phase, pouring the organic phase into an aqueous solution of ammonium sulfate at a concentration of about 100 to about 400 mmol/L, stirring at a high speed, homogenizing, ultrasounding or extruding at a high pressure to form a blank liposome suspension;
alternatively, weighing HSPC, other lipids, cholesterol and long-circulating materials in amounts of formula, dissolving them in tert-butanol, lyophilizing, adding the resultant to an aqueous solution of ammonium sulfate having a concentration of about 100 to about 400 mmol/L to dispense and to form a blank liposome suspension;
b. the external medium of the blank liposome suspension obtained in step a is exchanged about 5 to about 30 times volume with pure water or aqueous solution of sucrose through a tangential flow ultrafiltration device, to remove ammonium sulfate of the external aqueous phase to establish ammonium sulfate gradient;
c. adding irinotecan hydrochloride into the blank liposome suspension obtained in step b, incubating the resultant at a temperature higher than the liposome phase transition temperature for 10 min-1 h for drug-loading; and
d. adding a buffer salt and a non-ionic surfactant in a solid form into the drug-loaded liposome suspension, stirring and dissolving, adjusting the pH to 5.5 to 7.5, to obtain an irinotecan hydrochloride composite phospholipid composition;
or replacing the external medium of the drug-loaded liposome suspension through a tangential flow ultrafiltration device with a pharmaceutically acceptable buffer, then adding a non-ionic surfactant, adjusting the pH to 5.5 to 7.5, to obtain an irinotecan hydrochloride composite phospholipid composition.

34. The preparation process according to claim 33, further comprising a step of:
adding a lyoprotectant after step d.

35. The preparation process according to claim 33, further comprising, after step d or after the addition of lyoprotectant:
sterilizing the resultant by filtration with microfiltration membrane to obtain a sterile formulation.

36. A method of treating a tumor in a subject, comprising:
administering the irinotecan hydrochloride composite phospholipid composition according to claim 17 to the subject;
wherein the tumor is selected from the group consisting of colorectal cancer, non-small cell lung cancer, ovarian cancer, cervical cancer, stomach cancer, malignant lymphoma, breast cancer, skin cancer, and pancreatic cancer.
